(19) United States
(12) Patent Application Publication
(10) Pub. No.: US 2008/0319422 A1
(43) Pub. Date: Dec. 25, 2008

(54) EPIDURAL ANESTHETIC DELIVERY SYSTEM

(76) Inventor: Juan Cardenas, Carmel, IN (US)

Correspondence Address: INDIANO VAUGHAN LLP., SUITE 1300, ONE NORTH PENNSYLVANIA STREET, INDIANAPOLIS, IN, 46204, US

(21) Appl. No.: 12/143,228

(22) Filed: Jun. 20, 2008

Related U.S. Application Data
(60) Provisional application No. 60/999,257, filed on Oct. 17, 2007.
(60) Provisional application No. 60/936,607, filed on Jun. 21, 2007.

(51) Int. Cl. A61M 25/18

(52) U.S. Cl. 604/537

(57) ABSTRACT

A connector is provided for use in an anesthesia delivery system for coupling an anesthesia dispensing container to an anesthesia delivery catheter. The connector includes a first connector member and a second connector member. The first connector member includes a body member having a proximal end that is capable of being operably coupled to an anesthesia dispensing container. The first connector member also includes a distal end capable of being selectively coupled to the second connector member. The body further includes a fluid passageway having a proximal end and a distal end. The fluid passageway extends between the proximal end and the distal end of the body member. A check valve is disposed in the fluid passageway. The check valve is configured to: (a) permit the flow of fluid between the distal end and the proximal end of the fluid passageway regardless of whether the first connector member is coupled to the second connector member; (b) permit the flow of gas between the proximal end and the distal end of the fluid passageway regardless of whether the first connector member is coupled to the second connector member; and (c) permit the flow of fluid between the proximal end and the distal end of the fluid passageway only when the first connector member is coupled to the second connector member.

### I. RELATED APPLICATION

The application claims benefit of Cardenas, U.S. Provisional Patent Application No. 60/999,257 that was filed on 17 Oct. 2007; and Cardenas, U.S. Provisional Patent Application No. 60/936,607 that was filed on 21 Jun. 2007; both of which are fully incorporated herein by reference.

### I. TECHNICAL FIELD OF THE INVENTION

The present invention relates to a medical device for administering anesthetic and other medicines, and more specifically to a device for administering epidural anesthesia and providing a check against human error.

### II. BACKGROUND

Epidural anesthesia is a common form of anesthesia used for lower abdominal, pelvic, and lower extremity surgery. It is also commonly used for mothers during childbirth.

Epidural anesthesia is generally administered by inserting an epidural catheter into the epidural space located just outside the spinal cord. A standard syringe is connected to the catheter. Anesthetic agent contained within the barrel of the standard syringe is then expunged out of the syringe through the catheter and into the epidural space, flooding the nerves as they emerge from the spinal cord.

Patients receiving epidural anesthesia almost always receive fluids and other medications, including other anesthetics, intravenously in addition to the epidural anesthetic. In order to supply such intravenous materials, an intravenous catheter is inserted into a convenient vein. A syringe or drip line is then coupled to the intravenous catheter so that the material within the syringe and/or drip line can flow through the intravenous catheter and into the chosen vein.

The syringes used for both the epidural anesthetic and the intravenous fluid delivering are usually “standard” syringes. As such, the same type of syringes, having the same appearance are used for the administration of both intravenous fluids and epidural anesthesia. Since the same types of syringes are used for both procedures, it is easy for a practitioner to confuse a syringe containing an epidural anesthetic with one containing an intravenous fluid.

Unfortunately, because anesthesiologists and nurse anesthetists are human, accidents have occurred where a syringe containing an intravenous medication has been connected to an epidural catheter and vice versa, thereby mistakenly administering an intravenous medication into an epidural space instead of an anesthetic, and vice-versa. Should a mistake occur, the consequences can be life threatening, including severe neurological and cardiovascular problems, and in the most severe cases, death.

The Applicant attempted to reduce or eliminate the potential of human error caused by mistakenly switching epidural and intravenous syringes in his previous invention disclosed by Cardenas, U.S. Pat. No. 5,616,133. The '133 patent incorporates a check valve into a syringe. The check valve will only allow the discharge of the contents of the syringe in combination with the epidural catheter connector. While Applicant's previous invention performs its function in a sterling manner, room for improvement exists.

The incorporated syringe and check valve device disclosed in the '133 patent is a stand alone device with specific uses. As the '133 device was a customized, non-standard syringe, it suffered the defect of requiring hospitals and doctors to maintain multiple types of syringes in their inventory. Applicant's previous device is not compatible with currently existing syringes or currently existing catheters. As such, its usefulness is limited and its costs are higher than the cost of a standard syringe, since a separate syringe was required for a single application.

Because of the above problems, Applicant invented the present invention. The present invention functions as an attachment compatible with all standard syringes. When the present invention is connected to a syringe, it provides a check valve to prevent the injection of the epidural anesthetic unless connected to the epidural catheter, thereby limiting the likelihood of of human error resulting in an adverse outcome to a patient.

Also, as disclosed in the prior Cardenas patent, the distal end of the epidural catheter was directly coupled to the proximal end of the epidural catheter in a manner that prevented a filter from being used. Therefore, in the present invention, the Applicant has invented a “Secondary (epidural) catheter connector” that enables the present invention to be used in conjunction with a standard filter, or to be connected to one of many already existing standard (or primary) epidural catheter connectors.

One object of the present invention is to provide a check valve that prevents the epidural and intravenous medication from getting mixed up through human error. Another object of the present invention is to provide an attachment capable of use with any currently existing syringe and epidural catheter connectors.

### III. SUMMARY OF THE INVENTION

In accordance with the present invention, a connector is provided for use in an anesthesia delivery system for coupling an anesthesia dispensing container to an anesthesia delivery catheter. The connector comprises a first connector member and a second connector member. The first connector member includes a body member having a proximal end that is capable of being operably coupled to an anesthesia dispensing container. The first connector member also includes a distal end capable of being selectively coupled to the second connector member. The body further includes a fluid passageway having a proximal end and a distal end. The fluid passageway extends between the proximal end and the distal end of the body member. A check valve is disposed in the fluid passageway.

The check valve is configured to: (a) permit the flow of fluid between the distal end and the proximal end of the fluid passageway regardless of whether the first connector member is coupled to the second connector member; (b) permit the flow of gas between the proximal end and the distal end of the fluid passageway regardless of whether the first connector member is coupled to the second connector member; and (c) permit the flow of fluid between the proximal end and the distal end of the fluid passageway only when the first connector member is coupled to the second connector member.

The purpose of the present invention is to provide a method for eliminating human error in epidural injections as described above. The safe epidural system of the present invention provides a system that is installable on any existing syringe and, once installed, prevents the injection of the epidural substance unless the syringe fitted with the present invention is connected to a special epidural connector. When the present invention is connected to a syringe, an anesthesiologist cannot inject an anesthetic into a body tissue or intravenous line that does not contain the special epidural catheter connector.

The present invention also incorporates special connectors that prevent the injection of substances from a standard syringe into the epidural space and a tubing connector which allows continuous infusions to be made into the epidural catheter.

Compared to the most typically used prior art epidural delivery systems, the present invention provides a much safer epidural delivery system while increasing ease of use and compatibility with presently used syringes and other equipment.

### IV. BRIEF DESCRIPTIONS OF THE DRAWINGS

FIG. 1A is a side view of a prior art syringe used in epidural injections;

FIG. 1B is a side view of the syringe coupled to the dispensing connector of the present invention;

FIG. 2 is a side view of a standard syringe coupled to the dispensing connector and the secondary catheter connector of the present invention;

FIG. 3 is a side view of the connection components of the present invention;

FIG. 4 is a partially sectional view of the epidural system of the present invention;

FIG. 5A is a side view of the dispensing connector and the continuous epidural tubing being shown as being enjoined;

FIG. 5B is a side view showing the distal end of a continuous epidural tubing line being coupled to the proximal end of a dispensing connector of the present invention;

FIG. 5C is a side view of a complete assembly of the present invention wherein the continuous epidural tubing, dispensing connector, secondary epidural connector, filter, primary catheter connector and epidural catheter line are all coupled together;

FIG. 5D is a side view similar to FIG. 5C, with the filter removed;

FIG. 6 is an enlarged side, partially sectional view of the dispensing connector taken along lines 6-6 of FIG. 5A;

FIG. 7 is a side sectional view of the secondary catheter connector;

FIG. 8 is a sectional view partly broken away of the check valve portion of the dispensing connector;

FIG. 9 is a side view of a secondary epidural catheter connector that schematically illustrates that which occurs when one mistakenly attempts to use an IV syringe instead of an epidural syringe with the present invention; and

FIG. 10 is a side view of a secondary epidural catheter of the present invention wherein an unsuccessful attempt is being made to use a standard, needle-less syringe with the present invention.

### V. DETAILED DESCRIPTION

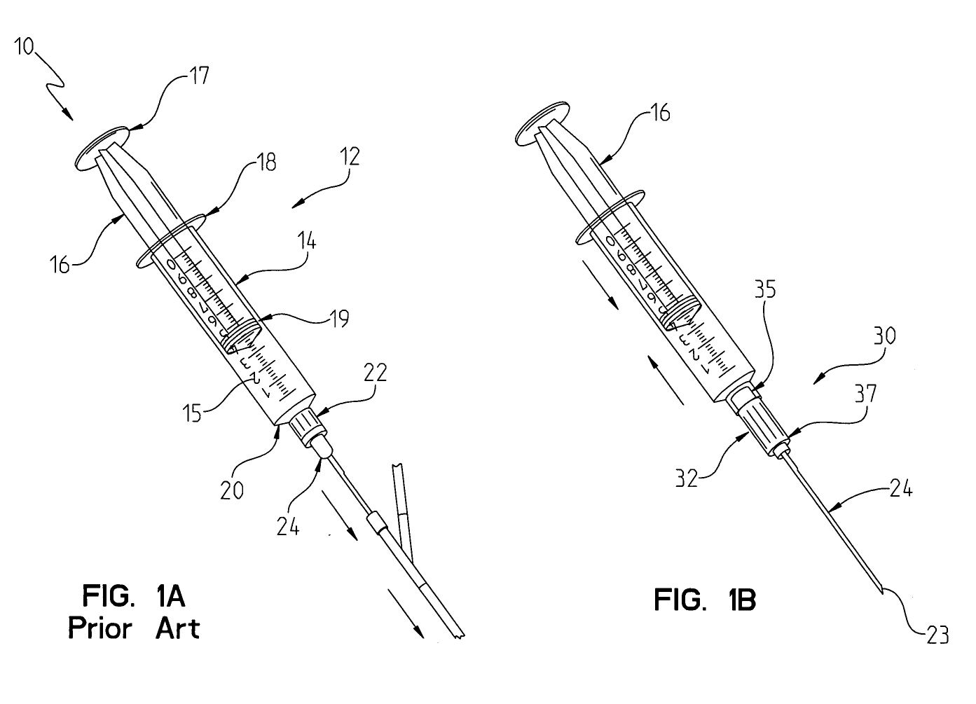

As best shown in FIG. 1A, prior art epidural delivery system 10 employed an anesthesia dispensing container that comprised a syringe such as standard syringe 12. The standard syringe 12 includes a hollow body 14 having a hollow interior cavity 15 and a plunger 16 having a first or proximal end 17 and a second (distal) end 19. First end 17 protrudes from the first (proximal) end 18 of the syringe 12. The second end 19 of plunger 16 is insertable into and normally resides within the hollow interior cavity 15 of body 14. The plunger 16 is movable axially within the hollow body 14 between a retracted position where the distal end 19 of the plunger 16 is positioned close to the proximal end 18 of the body 14; and an inserted position where the distal end 19 of the plunger 16 is positioned closer to the distal or second end 20 of the body 14 of the syringe 12.

Moving the plunger 16 from the inserted position toward the retracted position will create a reduced pressure in the hollow cavity 15 that will enable the syringe to draw fluid, such as a medication, into the syringe 12 cavity 15. Conversely, moving the plunger 16 from the retracted position toward the inserted position will tend to force whatever fluid (or gas) is contained within the cavity 15 out of the cavity 15, and through the distal end of a needle 24 attached to the syringe, to thereby expunge the fluid from the cavity 15.

The second (distal) end 20 of the syringe 12 includes a reduced diameter gripping portion 22 that is designed for being coupled to a proximally disposed coupling head of a hypodermic needle assembly 24. Needle assembly 24 includes a hollow interior passageway through which fluid can flow, and a sharpened distal end 23 designed for piercing tissue such as the skin. Needle 24 is designed for dispensing fluid into and out of the syringe 12.

Standard syringes 12 are inexpensive, easy to use and mass produced by the millions, if not the billions. Unfortunately, standard syringes 12 offer little protection from certain types of human error.

Many surgeries or medical procedures require multiple injections of different medicines in different places from different syringes. Often it is difficult to differentiate between different medicines in different syringes just by looking at the syringe, or even after reading their labels. This difficulty in differentiating leads to infrequent but avoidable mistakes wherein the wrong syringe is used for a particular task, which results in the wrong material being given to the patient. As discussed above injecting the wrong material into a patient (or more precisely, delivering a right material to the wrong location) can result in seriously adverse consequences to the patient.

The shortcomings of the prior art syringes led Applicant to create the present invention. The safe epidural system 30 of the present invention incorporates many of the parts of the standard syringe, including the body 12, the plunger 14 and the gripping portion 22.

A significant improvement of the epidural system 30 of the present invention is the introduction of dispensing connector 32 (FIG. 1B). The dispensing connector 32 is a device that is inexpensive to produce and includes a proximal end 35 and a distal end 37. The proximal end 35 of the dispensing connector 32 can be coupled easily onto the distal end (gripping portion 22) of standard syringe 12 by means of a standard Luer lock connection.

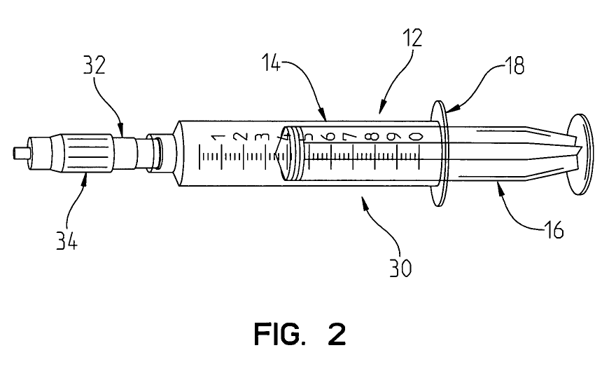

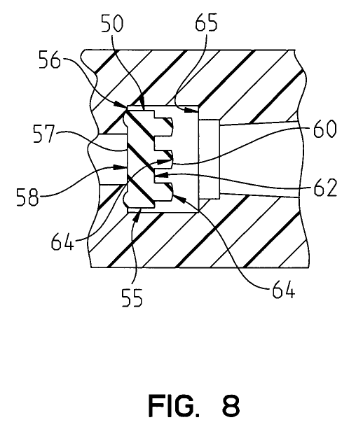

The distal end 37 of the dispensing connector is coupled to the secondary catheter connecter 34, providing a greatly improved method of eliminating human error in many surgeries (FIG. 2)¹. The dispensing connector 32 is attached to the gripping portion 22 of a standard syringe 12, and incorporates a one way check valve 50 (FIG. 8) allowing the user to draw fluid into the interior cavity of the body 14 of the syringe 12 but not to dispense fluid out of the body 14 interior, once drawn into the syringe 12. ¹For purposes of this application, we will refer to the already existing (standard) epidural catheter connectors as “primary epidural (catheter) connectors” and the connector 34 of the present invention as a “secondary epidural (catheter) connector”.

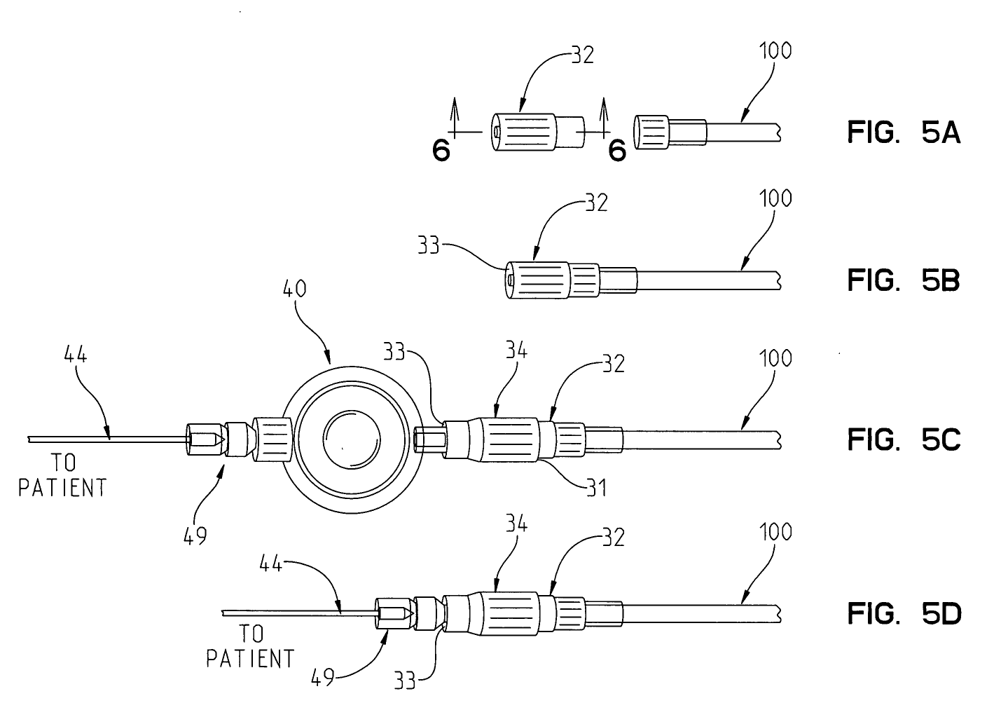

As shown in FIGS. 5A-5D, the secondary catheter connector 34 includes a proximal end 31 and a distal end 33. The proximal end 31 is sized and configured for being matingly removably coupled to the distal end 37 of the dispensing connector 32. The distal end 33 of the secondary epidural catheter connector 34 is sized and configured for being removably matably coupled to an injection component, such as filter 40 and/or primary epidural catheter connector 49.

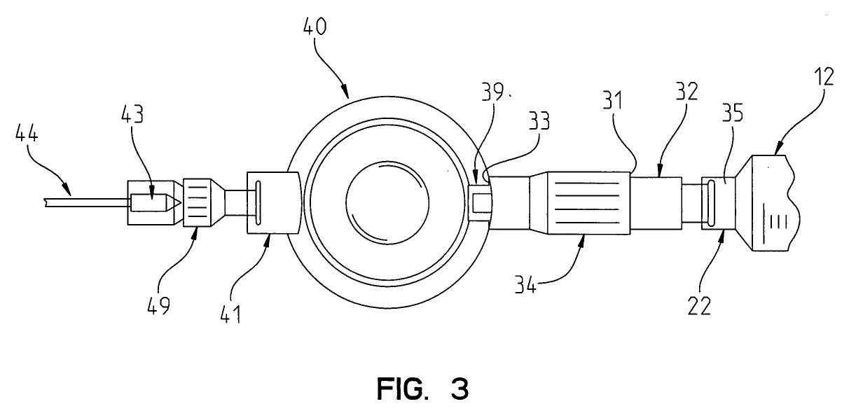

FIG. 3 shows a primary catheter connector 49 with female threaded portion 43 for interiorly threadedly receiving the male threads formed on the exterior surface of the distal end of the epidural catheter 44. A standard filter 40 is shown as being connected between, and in fluid communication with the primary epidural connector 49 and the secondary epidural catheter connector 34. The filter is connected to the primary catheter connector 49 via a distal Luer lock type connector 41. The secondary epidural connector 34 is connected to the proximal end of the filter 40 by a proximal Luer lock connector 39.

FIG. 3 illustrates the fully assembled device.

It will be noted that the epidural catheter 44 is located distally of the primary epidural connector 49, which itself is disposed distally of, and attaches via distal Luer lock 41 to standard filter 40. The standard filter 40 is connected by proximal Luer lock 39 to the secondary epidural catheter connector 34.

Although not shown, normal procedure would be to connect the proximal end 35 of the dispensing connector 32 to the distal end gripping portion 22 of syringe 12. The dispensing connector 32 containing syringe 12, is then coupled to the secondary epidural connector 34. Prior to the dispensing connector 32 and syringe 12 being coupled to the secondary epidural connector 34, the secondary epidural connector 34, along with filter 40, is already coupled to the epidural catheter 44.

When all of the components are so connected as shown in FIG. 3, the epidural anesthetic or other medicine contained with the syringe 12 can be administered to the patient by moving the plunger 16 of syringe 12 in an axially distal direction, to force fluid contained with the hollow body cavity 15 of the syringe 12 out the distal and of the syringe 12 and into the dispensing connector 32, and into and through the secondary epidural catheter connector 34, filter 40, primary epidural catheter connector 49, and then into epidural catheter 44.

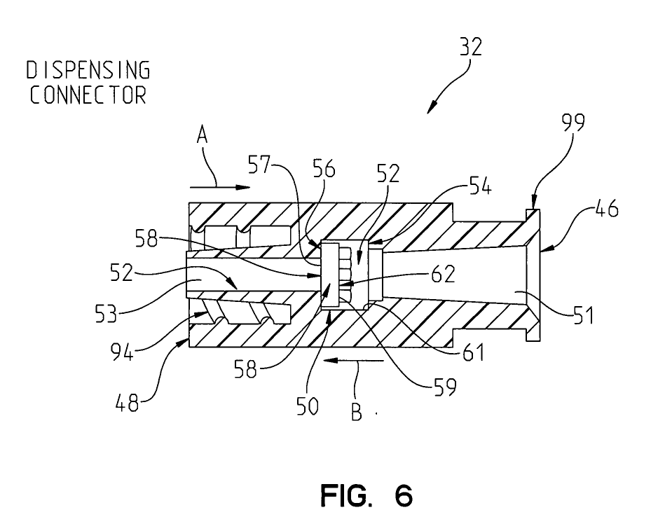

As best shown in FIG. 6, the dispensing connector 32 features a proximally disposed syringe connection end 35 and a distally disposed secondary epidural connector end 48. An axially extending passageway 52 extends between the proximal end 35 and the distal end 48. The axially extending passageway 52 includes a relatively reduced diameter proximal portion 51 disposed adjacent the proximal end 35, and a relatively reduced diameter distal portion 53 disposed adjacent the distal end 48. A relatively enlarged diameter valve containment chamber 54, which contains a movable valve-element (stopper) 58 is disposed between the reduced diameter proximal portion 51, and the reduced diameter distal portion 53.

The moveable valve element 58 and enlarged diameter portion 54 co-operate to form a check valve assembly 50. Check valve 50 allows liquid to be drawn in a direction indicated by arrow A into the syringe 12. For example, epidural anesthetic fluid may be withdrawn from a vial into the syringe 12 in direction A.

The valve 50 prevents expulsion of liquid in a direction indicated generally by arrow B unless all of the components of the system are present, and the valve element 58 is positioned within enlarged diameter portion 54 such that the element 58 is not positioned against the distally disposed shoulders 56 of the enlarged diameter portion 54, as shown in FIG. 6. Rather, the valve element 58 should be spatially separated from the distal shoulders 56, so that fluid within the syringe 12 traveling in direction B (proximal to distal) can flow around the valve element 58 and into distal portion 53 of passageway 52.

The check valve 50 preferably comprises an axially extending, hollow, relatively enlarged diameter cavity 54 formed within the dispensing connector's axially extending passageway 52. The check valve assembly 50 also includes an axially moveable stopper 58 that is disposed within the enlarged diameter portion 54. As shown in FIG. 8, the stopper 58 is generally cylindrical, featuring an axially facing, radially extending proximal surface portion 60 and an axially facing, radially extending distal surface portion 57. As best shown in FIGS. 6 and 8, the proximal surface 60 includes a plurality of grooves 62 carved into the proximal surface 60, and extending into side surface 55, forming several feet or legs 64 on the proximal portion 60 of the stopper 58. The distal portion 57 is generally planar and smooth, and may have a small, peripherally located ring as to maximize seal between distal surface 57 of stopper 58 and the distal wall 56 of enlarged diameter portion 54 of cavity 52. The stopper 58 also includes a generally cylindrical radially outwardly facing, axially extending side wall portion 55.

The axially proximal movement of plunger 16 to withdraw it from the interior cavity 15 of the body 14 increases the volume of space in cavity 15 capable of receiving material. The axial, proximal movement of plunger 16 causes liquid to be drawn into the syringe 12 cavity 15. The proximal surface 60 of the stopper 57 is concurrently pulled axially and proximally toward the proximal wall 61 of the enlarged diameter portion 54 of the cavity 52 so that the proximal surface 60 of the stopper 58 engages the radially extending, axially distally facing surface 61 of the enlarged diameter portion 54 of the stopper cavity 52. Even with valve head surface 60 engaging passageway 52 surface 61, fluid can still flow from the distal portion 53, cavity 52 around stopper 58 and into proximal portion 51 of cavity 52, and ultimately into the syringe 12, since the chordal grooves 62 formed on surface 60 of the stopper 58 allow liquid to pass around the stopper 58 and into the into the syringe 12.

When the syringe 12 is filled with liquid, the pressure of the liquid within the syringe 12 forces the planar distal surface 57 of the stopper 58 against the distal wall 56, forming a fluid tight seal, which prevents fluid from flowing in an axially distal direction (Arrow B, FIG. 6), thereby preventing the syringe 12 connected to the dispensing connector 32 from expelling the liquid through the dispensing connector 32.

If the syringe 12 is turned to a vertical position with the distal end of the hypodermic needle 24 pointed upward, and with the dispensing connector 32 pointed upwards, the check valve 50 will permit air and other gasses to escape the interior cavity 15 of the syringe 12 when plunger 16 is pushed axially distally in a slow and gentle fashion. This occurs because air density is lower than the density of stopper 58. However, even in this vertical position, the fluid pressure of the fluid in the syringe 12 cavity 15 will cause the stopper 58 to be pushed against the distal wall 56, forming a seal that prevents any liquid from passing distally through and out of the system.

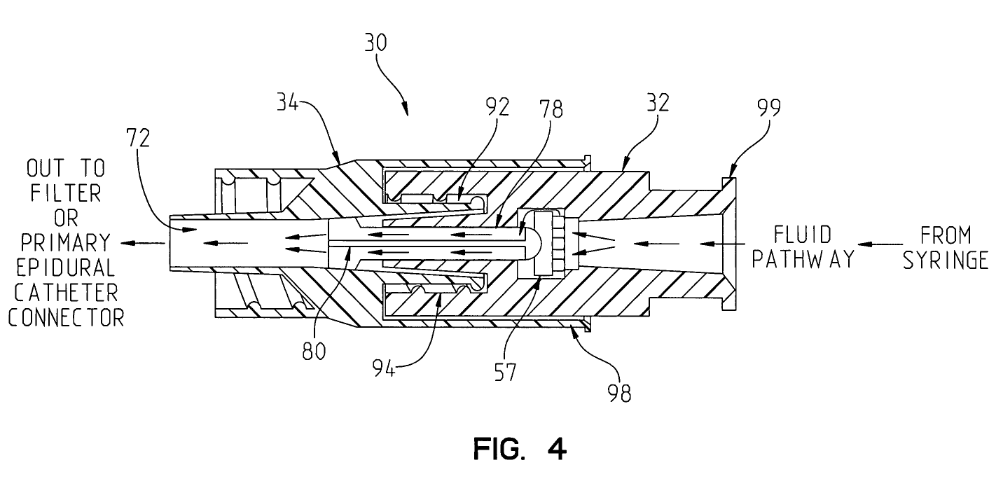

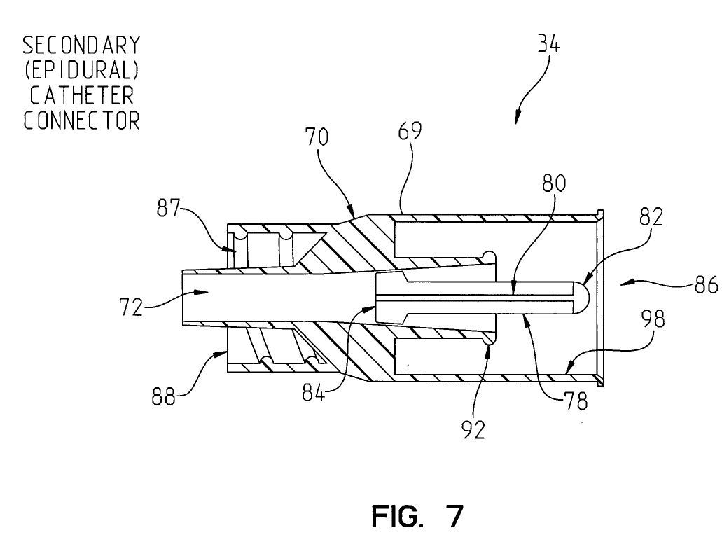

When the dispensing connector 32 is connected to the secondary epidural connector 34, the safe epidural system of the present invention 30 allows the epidural anesthetic or other medicine to be dispensed from the hollow interior 15 of the body 14 of the syringe 12. As best shown in FIGS. 4 and 7, the secondary epidural connector 34 includes a body 70 having a generally cylindrical outer surface 69. The secondary epidural connector 34 has a relatively enlarged diameter proximal end 86 that mates co-axially with the distal end 48 of the dispensing connector 32. The secondary connector 34 also includes a relatively reduced diameter distal end 88 which preferably comprises a Luer Lock connection 87. Luer Lock connector 87 is designed to mate with a standard filter 40 or a primary epidural catheter connector 49.

A passageway 72 extends axially throughout the length of the body 70 between the proximal end 86 and the distal end 88. Anesthesia can flow through the passageway 72 for allowing the device to inject anesthetic, once the system 30 is fully connected. When the secondary connector 34 is co-axially coupled to the dispensing connector, passageway 70 and passageway 52 form a continuous passageway through which anesthesia can flow from the cavity 15 of the syringe 12 to the filter 40 (if used) or epidural catheter 44.

An axially extending push rod 78 is fixedly coupled to, or formed as a part of secondary catheter 34. Push rod 78 is centrally disposed within passageway 72 to be coaxial with passageway 72. The push rod 78 extends primarily within the proximal portion of the passageway 72, and includes a series of axially extending vanes 80 having spaces therebetween through which fluid can pass. The push rod 78 has a hemispherical proximal end 82 that is aperture free and can serve as a fluid banner. The push rod 78 also includes a distal end 84 that rests approximately midway in passageway 72.

The hemispherical end portion 82 of the push rod 78 is capable of extending into the valve cavity 52 of the dispensing connector and engaging the distal surface 57 of the stopper 58 when the secondary epidural connector 34 is matingly engaged with the dispensing connector 32 as best shown in FIG. 4. When the secondary epidural connector 34 is engaged with the dispensing connector 32, the push rod 78 forces the stopper 58 into engagement with the proximal wall 61 of the enlarged diameter portion 54 of the valve cavity 52, allowing fluid to pass into the distal portion 53 of the cavity 52 of the dispensing connector 32 by passing through and around the chordal grooves 62 of the stopper 58.

The dispensing connector 32 and the secondary epidural connector 34 connect to each other through a luer lock mechanism, featuring a male luer lock portion 92 and a female luer lock portion 94, similar to luer locks conventionally used in standard syringes 12 and other similar applications. The male Luer Lock mechanism 92 is formed as a part of secondary connector 34. The male Luer Lock 92 is disposed within passageway 92 and is coaxial with the primary axis of the secondary epidural connector 34. Female Luer Lock 94 is disposed on the distal end 48 of the dispensing connector 32.

In order to couple the syringe 12, the dispensing connector 32, and the secondary catheter connector 34, the proximal end 46 of the dispensing connector 32 is first brought into contact with the mating portion 22 of the syringe 12, via another luer lock connection 99 to matingly engage this dispensing connector 32 with the syringe 12. An axially extending sleeve 98 on the secondary catheter connector 34 (FIG. 4) is sized and positioned for interiorly receiving the distal end 48 of the dispensing connector 32 to thereby help to guide the end 48 of the dispensing connector 32 into a mating engagement with the proximal end 86 of the secondary epidural connector 34. As the syringe 12, dispensing connector 32 and secondary connector 34 are brought together, they are held snugly, forming a fluid-tight seal so that fluid passing between the syringe 12, the dispensing connector 32 and the secondary epidural connector 34 does not leak from the components.

The male Luer lock portion 92 of the secondary catheter connector 34 connects to the female Luer lock portion 94 of the dispensing connector 32 in a similar manner. When the distal end 48 of the dispensing connector 32 is connected to the proximal end 86 of the secondary catheter connector 34, the hemispherical end 82 of the push rod 78 connects with the first (distal) end surface 57 of the stopper 58, thereby holding the stopper 58 in a spaced relation from the valve wall 56 and maintaining fluid communication between the hollow body 14 cavity 15 of the syringe 12 and the fluid passageway 72 of the secondary catheter connector so that fluid within the syringe 12 cavity 15 can be introduced into the secondary catheter connector 34, and ultimately into the body part (e.g. epidural space) into which the fluid (e.g. anesthetic) is intended to be delivered.

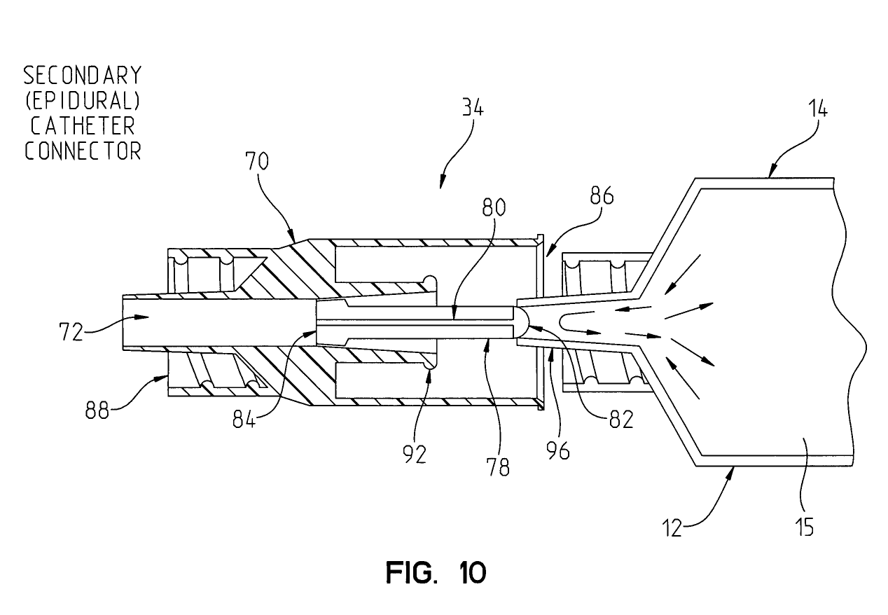

It should be noted that the inside diameter of the distal portion 53 of the axial passageway 52 of the dispensing connector 32 is larger than the inside diameter of the slip tip portion 96 (FIG. 10) of the Luer lock end of a standard syringe 12. This permits the push rod 78 to penetratingly enter and reach the valve member 58 when the dispensing connector 32 is connected to the secondary catheter connector 34. The wall thickness of the distal portion 53 of the axial passageway 52 of the dispensing member 32 is also less than that of the slip tip 96 of the Luer lock (FIG. 10) end of a standard syringe 12, which is necessary to allow the luer portions 94 of the dispensing connector 32 to engage or receive a hypodermic needle 24. The dispensing connector 32 must be able to accept a needle in its distal end 37 (FIG. 1B) so as to draw fluids into the syringe 12 once plunger 16 is pulled outwards in a proximal direction.

When the syringe 12, dispensing connector 32 and secondary catheter connector 34 are properly coupled together, and the epidural anesthetic or other medicine is ready to be dispensed into the patient via the catheter 44, the luer lock connectors 92, 94 between the dispensing connector 32 and the secondary catheter connector 34 provide an airtight and fluid-tight seal throughout the system, so when the plunger is moved axially distally within the hollow interior 15 of the body 14 of the syringe 12, the liquid contained in the interior cavity 15 of the syringe 12 passes out of the syringe 12, and flows into the dispensing connector 32 and around the stopper 58 valve. The liquid then flows through the spaces between the vanes 80 of the push rod 78 and into the fluid passageway 72 of the secondary catheter connector 34, where it can then enter the standard filter 40, and the standard epidural catheter connector 49 where it can enter the epidural catheter 44 per se and ultimately the epidural space of the patient and flood the patient's nerve endings with anesthesia, thereby producing analgesia.

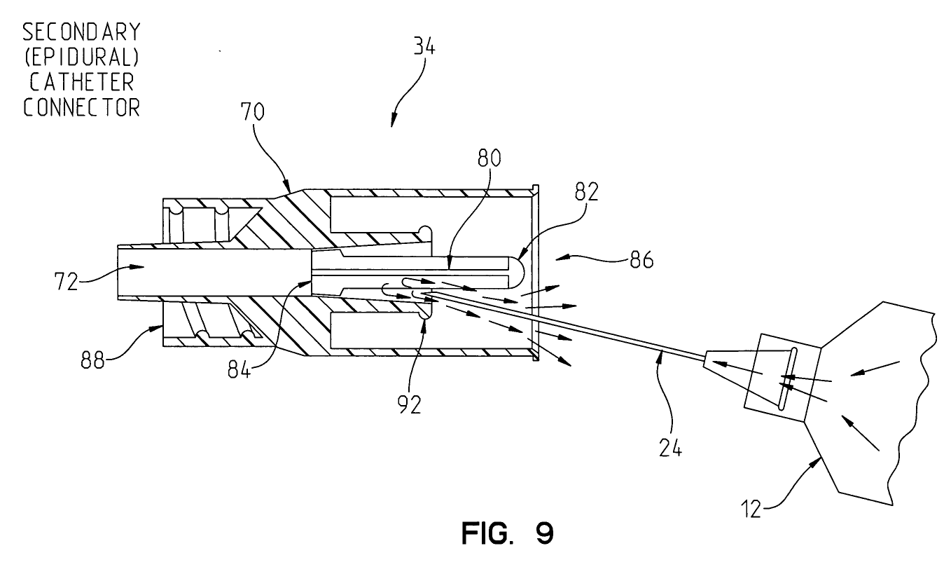

FIG. 9 illustrates the inability of standard needles to inject fluid through the secondary epidural catheter connector 34 and thus into the epidural space. In order for liquid to be injected through the connectors, there must be an airtight seal so fluid can be pushed through to the epidural space. If there is no seal, fluid will leak into the atmosphere and the fluid will not be able to exert enough pressure to travel through the connector 34 and epidural catheter 44 to thereby travel into the epidural space.

FIG. 9 also illustrates the mechanical incompatability between a standard hypodermic needle 24 and the push rod 78 containing proximal end 86 of the secondary catheter connector 34. By contrast, as discussed above, only the dispensing connector 34 has the mechanical configuration compatability with the secondary catheter connector 34 to enable fluid to flow appropriately through the secondary catheter 34.

FIG. 10 illustrates the inability of a standard syringe 12, with a distal Luer Lock mechanism to engage or mate with the proximal end 86 of the secondary epidural connector 34. The internal diameter of the slip tip 96 of the Luer Lock is smaller than the external diameter of push rod 78 and its end cap 82. As such, fluid exiting from the slip tip 96 of the syringe 12 is prevented by cap 82 from passing through the spaces between the vanes 80 of push rod 78. Therefore, fluid from the syringe 12 cannot enter passageway 72 or catheter 44. These features illustrated in FIGS. 9 and 10 prevent the accidental epidural injection of medications meant for intravenous or intramuscular use.

There are six primary steps involved in the use of the safe epidural system. First, the proximal end 46 of the dispensing connector 32 is attached to the distal end of the syringe 12. Preferably, the dispensing connector 32 is designed so that its connection to the syringe forms a “permanent” connection that discourages separation of the connector 32 from the syringe 12. Disconnection is discouraged since maintaining the connection between the syringe 12 and the dispensing connector 32 reduces the likelihood of the syringe being injected into an intravenous line.

After the dispensing connector 32 has been attached to the syringe 12, a needle 24 is attached to the distal end of the dispensing connector 32, and epidural anesthetic or other medicine is extracted from the container bottle in which it is packaged. Once the liquid has been extracted from its package, the stopper 58 of the dispensing connector 32 will not allow the epidural anesthetic or other medicine to exit the syringe 12 and enter into the needle 24. As such, the anesthetic-filled syringe can not be mistakenly used in an IV line, or dispensed into a body tissue, since even if an attempt is made to mistakenly use the syringe 12, the valve assembly 50 of the dispensing connector 32 will prevent the flow of fluid out of the syringe 12 and into the needle 24.

Nonetheless, air is easily removed from the syringe 12 when coupled to the dispensing connector 32. To remove air, the plunger 16 of the syringe 12 is slowly pressed in an axially distal direction while holding the syringe 12 upright so that the needle 24 is pointing straight up. When the liquid has been drawn into the interior cavity 15 of the body 14 of the syringe 12 and the air has been removed, the needle 24 is removed from the syringe 12, thereby forcing the stopper 58 of the valve 50 into a closed position as shown in FIG. 8.

The secondary catheter connector 34 is preferably then attached to the pre assembled epidural filter 40 and standard epidural connector assembly 49. In cases where a filter 40 is not used, the distal end 88 of the secondary catheter connection 34 is attached to the primary catheter connector 49.

A syringe 12 that is not fitted with the dispensing connector 32 will be incapable of dispensing its contents into the secondary catheter connector 34 as shown in FIGS. 9 and 10. The push rod 68 of the secondary catheter connector 34 prevents a syringe 12 from injecting medicine into the standard connector-filter-secondary connector assembly by completely blocking the passageway 72, as shown in FIG. 10.

Additionally, the system requires pressurization to dispense the syringe's 12 contents into the epidural catheter 44. Such pressurization can not be generated in the absence of every component of the present invention being properly connected together.

The dispensing connector 32 and syringe 12 are connected to the secondary catheter connector 34 and the remainder of the components including filter 40, primary catheter connector 49 and epidural catheter 44. The coupling of the dispenser 32 to the secondary catheter connector 34 causes the push rod 78 in the secondary catheter connector 34 to press against the distal surface of stopper 58 to thereby move the stopper 58 to an open position in the dispensing connector 32 (FIG. 4), thereby enabling the anesthetic to flow out of the syringe 12, past the stopper 58 and through the openings in the push rod 78, and into the passageway 72 of the secondary catheter connector 34, and ultimately through the filter 40, the primary epidural connector 49 and epidural catheter 44. The system can be used without standard filter 40.

Once the desired amount of fluid has been dispensed from the syringe 12 into the patient's epidural space, the dispensing connector 32 and syringe 12 are disconnected from the rest of the system. A second dispensing connector 32 attached to continuous epidural tubing 100 (FIG. 5) can now be attached to the secondary catheter connector 34 and allow desired fluid to continuously enter the epidural catheter 44.

The present invention provides mechanical devices which help the anesthesiologist and nurse anesthetist prevent the occurrence of human error, both by preventing the improper injection of medications intended for intravenous use into an epidural catheter and by preventing the improper injection of epidural anesthetic into a vein.

As stated above, this system is primarily designed to avoid accidental drug injections both into the epidural and intravenous spaces. However, equally as important are other circumstances in a medical practice where this system can be just as beneficial. For example, the present invention also has great utility for patients receiving single and/or continuous peripheral nerve blocks with highly cardiotoxic or neurotoxic local anesthetics. These patients have peripheral IV lines in place and therefore the same risk for human error exists. There may be other potential uses for this system not realized at this point in time.

Those skilled in the art will appreciate that other embodiments in addition to the embodiment described above exist, which fall within the scope and spirit of the invention, which is limited only by the prior art.

What is claimed is:

1. A connector for use in an anesthesia delivery system for coupling an anesthesia dispensing container to an anesthesia delivering catheter, the connector comprising a first connector member and a second connector member,
the first connector member including a body member having a proximal end capable of being operatively coupled to an anesthesia dispensing container, a distal end capable of being selectively coupled to the second connector member, a fluid passageway having a proximal end and a distal end, and extending between the proximal end and the distal end, and a check valve disposed in the fluid passageway, the check valve being configured to:
(a) permit the flow of fluid between the distal end and the proximal end of the fluid passageway regardless of whether the first connector member is coupled to the second connector member,
(b) permit the flow of gas between the proximal end and the distal end of the fluid passageway regardless of whether the first connector member is coupled to the second connector member; and
(c) permit the flow of fluid between the proximal end and the distal end only when the first connector member is coupled to the second connector member.

2. The connector of claim 1 wherein the anesthesia dispensing container comprises a syringe of the type that includes a distal end to which a hypodermic needle can be attached.

3. The connector of claim 2 wherein the proximal end of the first connector member is configured to matingly engage the distal end of the syringe to place the syringe in fluid communication with the fluid passageway of the first connector member.

4. The connector of claim 3 wherein the proximal end of the body member of the first connector member includes a coupling that permits the first connector member to be coupled to the syringe, but which discourages removal of the first connector member from the syringe.

5. The connector of claim 1 wherein the check valve is movable between a fluid dispensing position wherein fluid can flow from the proximal end to the distal end of the fluid passageway; and a non-dispensing position wherein fluid is prevented from flowing from the proximal end of the fluid passageway to the distal end of the fluid passageway.

6. The connector of claim 5 wherein the second connector member includes an actuator member capable of moving the check valve into the fluid dispensing position when the second connector member is coupled to the first connector member.

7. The connector of claim 6 wherein the actuator member includes an axially extending rod member fixedly coupled to second connector member.

8. The connector of claim 7 wherein the axially extending rod includes an end portion capable of engaging the check valve member and a side portion having at least one aperture therein through which fluid can flow.

9. The connector of claim 8 wherein the first connector member and the second connector member each include a Luer member for permitting the first connector member to be removably coupled to the second connector member.

10. The connector of claim 1 wherein the fluid passageway of the first connector member includes a reduced diameter distal portion, a reduced diameter proximal portion, and an enlarged diameter portion disposed between the distal portion and the proximal portion, a proximal portion opening between the enlarged diameter portion and the proximal portion, and a distal portion opening between the enlarged diameter portion and the distal portion.

11. The connector of claim 10 wherein the check valve includes a movable head member disposed with the enlarged diameter portion, the movable head member including a proximal surface capable of covering the proximal portion opening and a distal surface capable of covering the distal portion opening.

12. The connector of claim 11 wherein the distal surface of the valve head is capable of sealingly covering the distal opening for preventing the flow of fluid from the proximal portion to the distal portion, but wherein the valve head permits the flow of fluid from the distal portion to the proximal portion when the proximal surface of the valve head covers the proximal opening.

13. The connector of claim 12 wherein the valve head includes at least one groove formed in the proximal surface for permitting fluid to pass around the valve head and into the proximal portion.

14. The connector of claim 1 wherein the check valve includes a valve head member having a distal surface placed relatively closer to the distal end of the fluid passageway and a proximal surface disposed relatively closer to the proximal end, the proximal surface including at least one groove for permitting fluid to pass around the valve head and toward the proximal end.

15. The connector of claim 1 wherein the second connector includes a proximal end and distal end, the proximal end being capable of being coupled to the distal end of the first connector, and the distal end including a distal end connector for permitting the second connector to be coupled to a component of the anesthesia delivery system.

16. The connector of claim 15 wherein said component of the anesthesia delivery system is selected from the group consisting of a filter, a catheter, and a catheter connector.

17. An anesthesia delivery system comprising an anesthesia dispensing container capable of selectively dispensing a quantity of fluid anesthetic,
a catheter member capable of being inserted into a tissue of a patient,
a first connector member having a proximal end capable of being coupled to the anesthesia dispensing container, and a distal end, the first connector member including a fluid passageway extending between the proximal end and the distal end and including a proximal portion, a distal portion and a check valve disposed in the fluid passageway,
a second connector member having a proximal end capable of being selectively removably coupled to the first connector member, and a distal end capable of being connected to a component of the anesthesia delivery system, the second connector member including a fluid passageway therein capable of being placed in fluid communication with the fluid passageway of the first connector member,
wherein the check valve is configured to:
(a) permit the flow of fluid between the distal end and the proximal end of the fluid passageway of the first connector member regardless of whether the first connector member is coupled to the second connector member;
(b) permit the flow of gas between the proximal end and the distal end of the fluid passageway of the first connector member regardless of whether the first connector member is coupled to the second connector member; and
(c) permit the flow of fluid between the proximal end and the distal end only when the first connector member is coupled to the second connector member.

18. The anesthesia delivery system of claim 17 wherein the check valve is movable between a fluid dispensing position wherein fluid can flow from the proximal end to the distal end of the fluid passageway of the first connector member; and a non-dispensing position wherein fluid is prevented from flowing from the proximal end of the fluid passageway to the distal end of the fluid passageway.

19. The anesthesia delivery system of claim 18 wherein the second connector member includes an actuator member capable of moving check valve into the fluid dispensing position when the second connector member is coupled to the first connector member.
